(19) United States
(12) Patent Application Publication
(10) Pub. No.: US 2026/0257078 A1
(43) Pub. Date: Sep. 3, 2026

(54) METHOD OF RADIATING ULTRASOUND BEAM TO ACCELERATE LYMPHATIC CIRCULATION

(71) Applicant: Research & Business Foundation SUNGKYUNKWAN UNIVERSITY, Suwon-si (KR)

(72) Inventors: Jinhyoung PARK, Gyeonggi-do (KR); Taewon CHOI, Gyeonggi-do (KR); Seoyun CHANG, Gyeonggi-do (KR)

(73) Assignee: Research & Business Foundation SUNGKYUNKWAN UNIVERSITY, Suwon-si (KR)

(21) Appl. No.: 19/223,359

(22) Filed: May 30, 2025

(30) Foreign Application Priority Data
Feb. 28, 2025 (KR) 10-2025-0027028

(51) Int. Cl. A61N 7/00

(52) U.S. Cl. CPC A61N 7/00; A61N 2007/0082

(57) ABSTRACT

A method of radiating an ultrasound beam to accelerate lymphatic circulation may be provided. In detail, the method of radiating an ultrasound beam includes radiating a focused ultrasound beam into tissues of a human body while moving a position of the focused ultrasound beam, sweeping the focused ultrasound beam around an open area of a blood-brain barrier (BBB) inside the tissues of the human body, and activating circulation of lymph in a path through which waste materials in the tissues of the human body are discharged, by performing a sweeping operation of repeatedly scanning the focused ultrasound beam in a certain pattern.

### CROSS-REFERENCE TO RELATED APPLICATION

This application claims the benefit of Korean Patent Application No. 10-2025-0027028, filed on Feb. 28, 2025, in the Korean Intellectual Property Office, the disclosure of which is incorporated herein in its entirety by reference.

### BACKGROUND

### 1. Field

The disclosure relates to a method of radiating an ultrasound beam to accelerate lymphatic circulation. In detail, the disclosure relates to a technology for radiating a focused ultrasound beam to accelerate the circulation of lymph and discharge waste materials from the tissues of a human body.

This research was supported by the Samsung Future Technology Promotion Project.

### 2. Description of the Related Art

Lymph circulates inside a human body. Lymph may circulate through lymphatic vessels formed along the tissues of the human body. Lymph may supply nutrients to the tissues. Lymph may receive waste materials from the tissues. Lymph may remove the waste materials from the tissues by discharging the waste materials into the veins through inter-tissue circulation and lymphatic circulation.

As a concrete example to explain the function of lymph, the circulation of cerebrospinal fluid (CSF) that flows around and inside the brain of the human body may be introduced. The CSF may remove waste materials in the brain through the lymphatic (for example, glymphatic) system. The lymphatic system may be an organic system to remove waste materials generated due to metabolism in the central nervous system of vertebrates. The CSF may be generated in the choroid plexus located inside the brain of the human body. The CSF may move toward the subarachnoid space of the brain. The CSF may move into the brain through the perivascular space formed along the brain blood vessels and enter the brain through the aquaporin 4 channel (AQP4) in the membrane made of astrocyte terminals. The CSF may receive waste materials while circulating inside the brain. The CSF may discharge the waste materials into the venous system via the lymphatic vessels.

Various methods have been proposed to accelerate the lymphatic circulation in order to remove waste materials generated from the tissues of the human body. According to the related art, mechanical stimulation is transmitted from outside the skull to the surface of the brain through massaging of the neck or scalp. However, methods that transmit mechanical stimulation from outside the skull do not directly stimulate the interior of the brain and improve the circulation of CSF, and thus there is a question as to whether the methods have the effect of activating the lymphatic system.

Furthermore, according to the related art, treatment using drugs that induce sleep which uses the characteristics that the resistivity of lymphatic circulation decreases during sleep of the human body has been employed, or chemicals that may dilate blood vessels have been used. However, drug treatment performed to activate the lymphatic system through sleep induction, which is a chemical method, may increase the risk of side effects. Furthermore, sleep auxiliary devices (sleep pillows, eye masks, etc.) sold to improve sleep may have low effectiveness as the devices indirectly activate the lymphatic system.

Accordingly, a lymphatic circulation acceleration technology that directly stimulates the interior of the brain without using chemical methods has been proposed. Recently, there have been attempts to open the blood-brain barrier (BBB) and remove waste materials from the brain by scanning an ultrasound beam across the entire brain. However, scanning the entire brain with an ultrasound beam may result in side effects due to the opening of the blood-brain barrier over a wide area. Accordingly, there is a need for a method of radiating an ultrasound beam that can accelerate lymphatic circulation with reduced side effects.

### SUMMARY

According to an aspect of the disclosure, a method of radiating an ultrasound beam includes radiating a focused ultrasound beam into a brain while moving a position of the focused ultrasound beam, sweeping the focused ultrasound beam around an open area of a blood-brain barrier (BBB) inside the brain, and activating circulation of lymph in a path through which waste materials in the brain are discharged, by performing a sweeping operation of repeatedly scanning the focused ultrasound beam in a certain pattern.

According to an aspect of the disclosure, a method of radiating an ultrasound beam to accelerate lymphatic circulation includes radiating a focused ultrasound beam to a boundary portion between a skull and brain parenchyma, allowing the focused ultrasound beam to enter the boundary portion at a specific angle, radiating the focused ultrasound beam while moving a position of the focused ultrasound beam, and transmitting the focused ultrasound beam to an activation trigger area where a lymphatic system is activated, by performing a sweeping operation of repeatedly scanning the focused ultrasound beam in a certain pattern.

### BRIEF DESCRIPTION OF THE DRAWINGS

These and/or other aspects will become apparent and more readily appreciated from the following description of the embodiments, taken in conjunction with the accompanying drawings in which:

FIG. 1 is a diagram illustrating the concept of a method of radiating an ultrasound beam to accelerate lymphatic circulation, according to an embodiment;

FIG. 2 is a flowchart showing a method of radiating an ultrasound beam to accelerate lymphatic circulation, according to an embodiment;

FIG. 3 is a diagram showing a focused ultrasound beam radiated to the brain of a human body according to a method of radiating an ultrasound beam to accelerate lymphatic circulation, according to an embodiment;

FIG. 4 is a flowchart showing a method of radiating an ultrasound beam to accelerate lymphatic circulation, according to an embodiment;

FIG. 5 is a diagram showing a focused ultrasound beam radiated while changing the position of the focused ultrasound beam according to a method of radiating an ultrasound beam to accelerate lymphatic circulation, according to an embodiment;

FIG. 6 is a diagram showing removal of waste materials by sweeping a focused ultrasound beam according to a method of radiating an ultrasound beam to accelerate lymphatic circulation, according to an embodiment;

FIG. 7 is a diagram showing removal of waste materials by sweeping a focused ultrasound beam according to a method of radiating an ultrasound beam to accelerate lymphatic circulation, according to an embodiment;

FIG. 8 is a diagram showing patterns formed when performing a sweeping operation of repeatedly scanning a focused ultrasound beam, according to an embodiment; and

FIG. 9 includes graphs showing a comparison of circulation amount of lymph depending on the application of a method of radiating an ultrasound beam to accelerate lymphatic circulation, according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term "and/or" includes any and all combinations of one or more of the associated listed items. Expressions such as "at least one of," when preceding a list of elements, modify the entire list of elements and do not modify the individual elements of the list.

Various embodiment of the document and terms used therein are not intended to limit the disclosure to particular modes of practice, and it is to be appreciated that various modifications, equivalents, and/or alternatives that do not depart from the spirit and technical scope of the disclosure are encompassed in the disclosure.

Throughout the drawings, similar or relevant parts are referenced with similar reference numerals.

The singular form of the noun corresponding to an item may include one item or a plurality of items, unless the relevant context clearly dictates otherwise.

In the disclosure, the expressions such as "A or B," "at least one of A and/or B," or "at least one or more of A and/or B" may include all available combinations of items listed together. For example, the expressions such as "A or B," "at least one of A and B," or "at least one of A or B" may signify all cases of including at least one A, including at least one B, or including both of at least one A and at least one B.

The term "and/or" includes any and all combinations of one or more of the associated listed items.

Terms such as "1^{st}" and "2^{nd}" or "first" and "second" are used herein merely to distinguish one element from another element and do not limit the elements in another aspect (e.g., order or importance).

When a component (e.g., a first component) is referred to as being "coupled" or "connected" to another component (e.g., a second component), with or without the terms "functionally" or "communicatively," it means that the component can be connected to the other component directly (e.g., wired), wirelessly, or via a third component.

Terms such as "include" or "comprise" may be construed to denote a certain characteristic, number, step, operation, component, or a combination thereof, but may not be construed to exclude the existence of or a possibility of addition of one or more other characteristics, numbers, steps, operations, components, or combinations thereof.

When a component is said to be "connected," "coupled," "supported," or "in contact" with another component, this includes not only cases where the components are directly connected to, coupled to, supported by, or in contact with each other, but also cases where the components are indirectly connected to, coupled to, supported by, or in contact with each other through a third component.

When a component is referred to as being "on" another component, the component can be directly on another component or between two intervening elements.

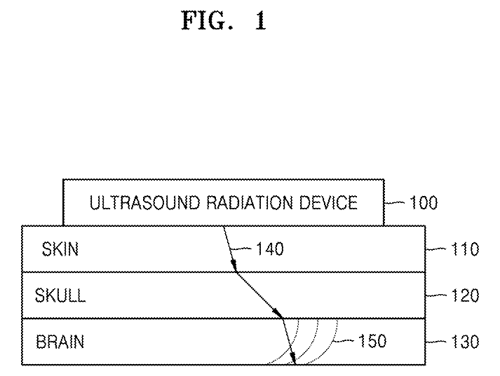

FIG. 1 is a diagram illustrating the concept of a method of radiating an ultrasound beam to accelerate lymphatic circulation, according to an embodiment.

The method of radiating an ultrasound beam to accelerate lymphatic circulation, according to an embodiment, may be performed by using an ultrasound radiation device 100. The ultrasound radiation device 100 may be a device that radiates an ultrasound beam in a specific direction. The ultrasound radiation device 100 may include an ultrasound converter to radiate an ultrasound beam in a specific direction. For example, the ultrasound radiation device 100 may include a mechanical or electronic ultrasound sweeper for radiating an ultrasound beam.

The ultrasound beam radiated from the ultrasound radiation device 100 may be incident on a skin 110. The ultrasound beam incident on the skin 110 may travel in a first direction. The ultrasound beam incident on the skin 110 may be incident on a skull 120. The ultrasound beam incident on the skull 120 may be refracted. The ultrasound beam incident on the skull 120 may travel in a second direction. The ultrasound beam incident on the skull 120 may be incident on a brain 130. The ultrasound beam incident on the brain 130 may be refracted. The ultrasound beam incident on the brain 130 may travel in a third direction. FIG. 1 expresses a case that the ultrasound beam is incident on the brain 130. However, this is not limited to this case, and the ultrasound beam may be incident on tissues of the human body.

For accelerating lymphatic circulation, the ultrasound radiation device 100 may radiate a first ultrasound beam 140 at a first angle. The first angle may be a preset angle for accelerating lymphatic circulation of the brain 130. The first angle may be an angle between a first axis perpendicular to the surface of the earth and a second axis along which an ultrasound beam (e.g., the first ultrasound beam 140) is incident. For example, the first angle may be 25° to 55°. The first angle may include a threshold angle for converting longitudinal waves into transverse waves. The threshold angle may be 30°.

The first ultrasound beam 140 that is radiated at the first angle may be incident on the brain 130 through the skin 110 and the skull 120. The first ultrasound beam 140 incident on the brain 130 may generate transverse waves 150 inside the brain 130. The transverse waves 150 generated inside the brain 130 may accelerate the lymphatic circulation of the brain 130. The transverse waves 150 generated inside the brain 130 may discharge waste materials in the brain 130.

The present disclosure provides an ultrasonic sweeping method for increasing the circulation speed of lymph by focusing and transmitting ultrasonic stimulation to a portion where lymphatic circulation is desired to be accelerated. According to the ultrasonic sweeping method according to the disclosure, the position of the ultrasonic stimulation may be moved by imitating a sweeping motion using a broom.

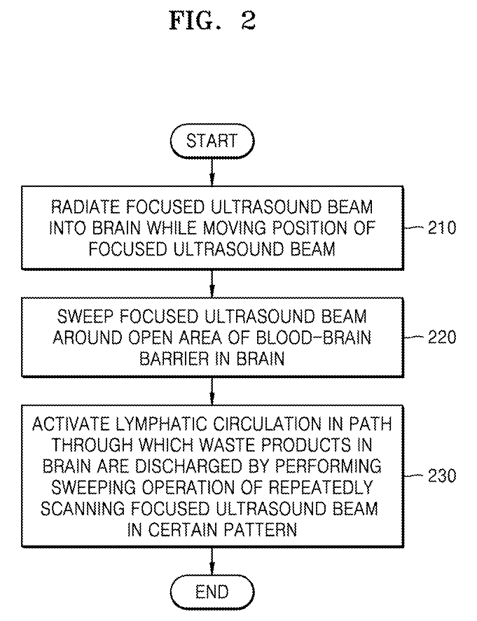

FIG. 2 is a flowchart showing a method of radiating an ultrasound beam to accelerate lymphatic circulation, according to an embodiment.

In operation 210, according to the method of radiating an ultrasound beam to accelerate lymphatic circulation, according to an embodiment, a focused ultrasound beam may be radiated into the brain while moving the position of the focused ultrasound beam. According to an embodiment, a focused ultrasound beam may be radiated to a region where the circulation of lymph flowing along lymphatic vessels formed along the tissues of the human body can be activated. For example, a focused ultrasound beam may be radiated to a region where lymphatic circulation in a path through which waste materials in the brain are removed can be activated. FIG. 2 expresses a case that the focused ultrasound beam is radiated into the brain. However, this is not limited to this case, and the focused ultrasound beam may be radiated into tissues of the human body.

In detail, the lymphatic (for example, glymphatic) system in the brain may remove waste materials from the brain through the circulation of cerebrospinal fluid (CSF). According to an embodiment, the lymphatic system may be activated by radiating an ultrasound beam so as to accelerate the circulation of CSF, thereby removal of waste materials in the brain. For example, the flow rate of lymph may be increased by radiating the ultrasound beam to have ultrasound focused on the subarachnoid sinus. When the flow rate of lymph increases, the pressure in the lymphatic vessels decreases, which may increase an amount of lymph flowing into the lymphatic vessels from the brain parenchyma.

In operation 220, according to the method of radiating an ultrasound beam to accelerate lymphatic circulation, according to an embodiment, the focused ultrasound beam may be swept around the open area of a blood-brain barrier (BBB) in the brain. According to an embodiment, the BBB may be open to a local area within the brain. According to an embodiment, the focused ultrasound beam may be swept around the open area of the BBB. For example, the sweeping may imitate a task of collecting wastewater around the open area of the BBB and discharging the wastewater into a sewer. In this case, the resistivity of waste materials discharged into the blood vessels of the open area of the BBB may be lower than that of the surrounding area. The discharge of waste materials within the brain may be increased by improving the movement of waste materials through a sweeping operation in the direction of decreasing the resistivity of discharge of waste materials in the open area. Furthermore, by performing sweeping in the reverse direction, the supply of compounds such as drugs flowing into the brain from the blood vessels may be increased.

In operation 230, according to the method of radiating an ultrasound beam to accelerate lymphatic circulation, according to an embodiment, by performing a sweeping operation of repeatedly scanning a focused ultrasound beam in a certain pattern, the lymphatic circulation in the path through which waste materials in the brain are discharged can be activated. According to an embodiment, a beam motion may be performed by scanning the focused ultrasound beam in a certain pattern. For example, the focused ultrasound beam may be scanned by reciprocating linearly. For example, the focused ultrasound beam may be scanned in a circular, oval, or arbitrary shape.

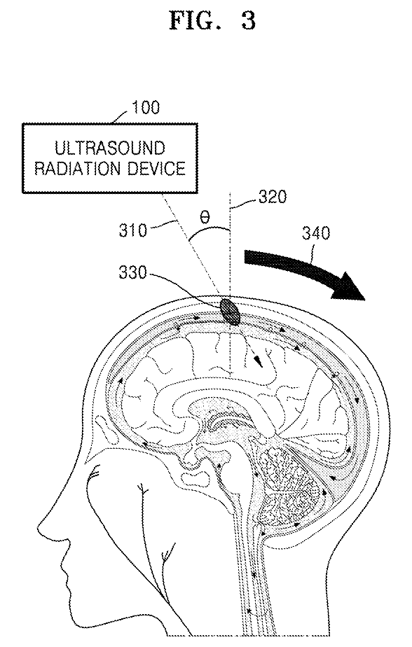

FIG. 3 is a diagram showing a focused ultrasound beam 310 radiated to the brain of a human body according to a method of radiating an ultrasound beam to accelerate lymphatic circulation, according to an embodiment.

The method of radiating an ultrasound beam to accelerate lymphatic circulation, according to an embodiment, may be performed by using the ultrasound radiation device 100. The ultrasound radiation device 100 may radiate the focused ultrasound beam 310 to the brain of a human body. The focused ultrasound beam 310 may be incident on the brain of a human body. FIG. 3 expresses a case that the focused ultrasound beam 310 is incident on the brain. However, this is not limited to this case, and the focused ultrasound beam 310 may be incident on tissues of the human body.

The focused ultrasound beam 310 may be scanned in burst form. The focused ultrasound beam 310 may be output in a certain pulse wave form. The circulation speed of lymph may be increased by concentrating and transmitting ultrasound brain stimulation to a portion where the lymphatic system can be activated.

The focused ultrasound beam 310 may be continuously radiated. The focused ultrasound beam 310 may be output in the form of a wave that has a constant size over a period of time. The circulation speed of lymph may be increased by concentrating and transmitting ultrasound brain stimulation to a portion where the lymphatic system can be activated.

The focused ultrasound beam 310 may be obliquely incident on the brain of a human body. The focused ultrasound beam 310 may be incident in a first direction. The focused ultrasound beam 310 may form a specific angle θwith a second direction 320 perpendicular to the surface of the brain. The specific angle θmay be 25° to 55°. For example, the specific angle θmay be 30°. The specific angle θmay be a threshold angle for converting longitudinal waves into transverse waves. A method of measuring the threshold angle may include two types of methods: a method of measuring an angle between a first direction in which the focused ultrasound beam 310 is incident and the second direction 320 perpendicular to the surface of the brain and a method of measuring an angle between the first direction and the surface (or bottom surface) of the brain. When an angle between the first direction and the surface of the brain is measured, the threshold angle may be measured to be 60°.

When the focused ultrasound beam 310 is scanned at the specific angle θ, transverse waves may be generated in the brain by the focused ultrasound beam 310. The generated transverse waves may accelerate the movement of lymph in the lymphatic vessels in the brain.

The focused ultrasound beam 310 may be in contact with the skull at an ultrasound focus point 330. The focused ultrasound beam 310 may be incident on the brain at the ultrasound focus point 330.

According to the method of radiating an ultrasound beam to accelerate lymphatic circulation, according to the disclosure, the focused ultrasound beam 310 may be radiated while moving the position of the focused ultrasound beam 310 in a direction indicated by an arrow 340. For example, the position of the focused ultrasound beam 310 may be moved along the venous sinuses in the brain in the direction indicated by the arrow 340. When the position of the focused ultrasound beam 310 is moved in the direction indicated by the arrow 340, the transverse waves generated by the focused ultrasound beam 310 may push lymph in the lymphatic vessels around the venous sinuses to accelerate the movement of lymph. For example, an interstitial fluid (ISF) introduced into the lymphatic vessels may be increased by the transverse waves generated by the focused ultrasound beam 310. For example, the CSF entering the brain parenchyma may be increased by the transverse waves generated by the focused ultrasound beam 310.

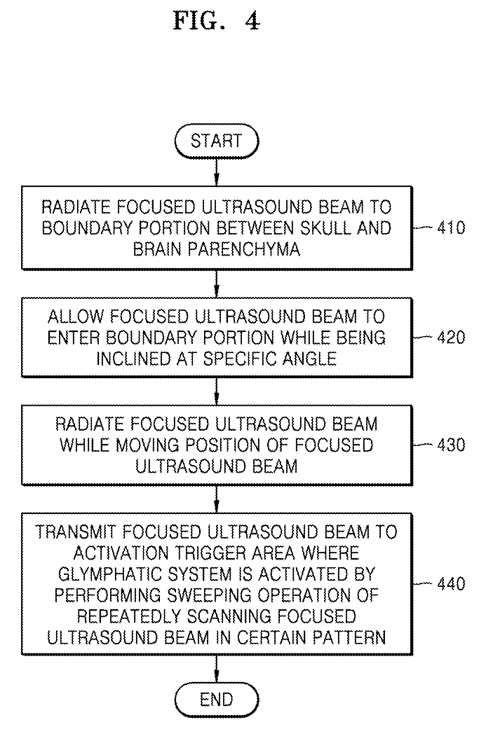

FIG. 4 is a flowchart showing a method of radiating an ultrasound beam to accelerate lymphatic circulation, according to an embodiment.

In operation 410, according to the method of radiating an ultrasound beam to accelerate lymphatic circulation, according to an embodiment, a focused ultrasound beam may be radiated to a boundary portion between the skull and the brain parenchyma. According to an embodiment, in order to transmit the ultrasound beam to a hotspot where the lymphatic system is activated, transverse waves may be generated by focusing ultrasound on the boundary portion between the skull and the brain parenchyma.

According to an embodiment, in order to transmit the ultrasound beam to the hotspot where the lymphatic system is activated, optimized acoustic scanning conditions for accurately focusing ultrasound energy on the boundary portion between the skull and the brain parenchyma may be set. To this end, a brain substructure may be fabricated where sweeping is performed using acoustic impedance values in an actual skull and soft tissues. Furthermore, when a target portion is swept by an ultrasound beam that is inclined by a specific angle, a flow of ultrasound energy propagating in the substructure may be simulated.

In operation 420, according to the method of radiating an ultrasound beam to accelerate lymphatic circulation, according to an embodiment, the focused ultrasound beam may enter the boundary portion between the skull and the brain parenchyma while being inclined at a specific angle. According to an embodiment, in order to generate transverse waves in the brain, when entering into the skull, the focused ultrasound beam may be inclined at a specific angle. The specific angle may be 25° to 55°. The specific angle may include a threshold angle for converting longitudinal waves into transverse waves. For example, threshold angle may be 30°. For example, it may be seen that, as a result of the focused ultrasound beam performing sweeping by setting an incident angle to be 30° from the surface of the skull, the focused ultrasound beam having the same directivity as a direction for sweeping at a portion directly under the skull is propagated in the brain substructure.

In operation 430, according to the method of radiating an ultrasound beam to accelerate lymphatic circulation, according to an embodiment, the focused ultrasound beam may be radiated while moving the position of the focused ultrasound beam. According to an embodiment, transverse waves may be generated in the brain by radiating the focused ultrasound beam while moving the position of the focused ultrasound beam.

In operation 440, according to the method of radiating an ultrasound beam to accelerate lymphatic circulation, according to an embodiment, by performing a sweeping operation of repeatedly scanning the focused ultrasound beam in a certain pattern, the focused ultrasound beam may be transmitted to an activation trigger area where the lymphatic system is activated. According to an embodiment, transverse waves may be generated in the brain by moving the focused ultrasound beam in a sweeping motion.

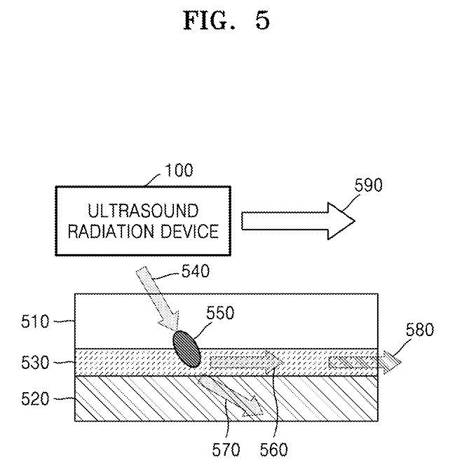

FIG. 5 is a diagram showing a focused ultrasound beam 540 radiated while changing the position of the focused ultrasound beam 540 according to a method of radiating an ultrasound beam to accelerate lymphatic circulation, according to an embodiment

The method of radiating an ultrasound beam to accelerate lymphatic circulation, according to an embodiment, may be performed by using the ultrasound radiation device 100. The ultrasound radiation device 100 may radiate the focused ultrasound beam 540. The focused ultrasound beam 540 may be incident on a first medium 510. Part of the focused ultrasound beam 540 incident on the first medium 510 may be refracted. The refracted ultrasound beam may be incident on a second medium 520. The rest of the focused ultrasound beam 540 incident on the first medium 510, except the refracted ultrasound beam, may travel along a fluid 530. For example, the fluid 530 may be lymph.

The focused ultrasound beam 540 incident on the first medium 510 may be refracted and separated at a focus point 550. The focus point 550 may be located at a boundary between the first medium 510 and the second medium 520. The focus point 550 may be located at the fluid 530.

A first beam 560 of the focused ultrasound beam 540 incident on the first medium 510 may travel along the fluid 530. The first beam 560 may have the characteristics of transverse waves. A second beam 570 of the focused ultrasound beam 540 incident on the first medium 510 may in incident on the second medium 520. The second beam 570 may have the characteristics of longitudinal waves. The focused ultrasound beam 540 incident on the first medium 510 may be split into the first beam 560 and the second beam 570, thereby having ultrasound radiation power with bending properties.

According to an embodiment, a force 580 for moving the fluid 530 may be generated by moving the ultrasound radiation device 100. In order to generate the force 580 that moves the fluid 530, the ultrasound radiation device 100 may be moved in a direction indicated by an arrow. Wave energy for moving the fluid 530 may be generated by the first beam 560 of the focused ultrasound beam 540 incident on the first medium 510. When the ultrasound radiation device 100 is moved in a direction indicated by the arrow 590, the incidence position of the ultrasound beam may be moved. When the incidence position of the ultrasound beam is moved, the force 580 of the first beam 560 moving the fluid 530 may be generated.

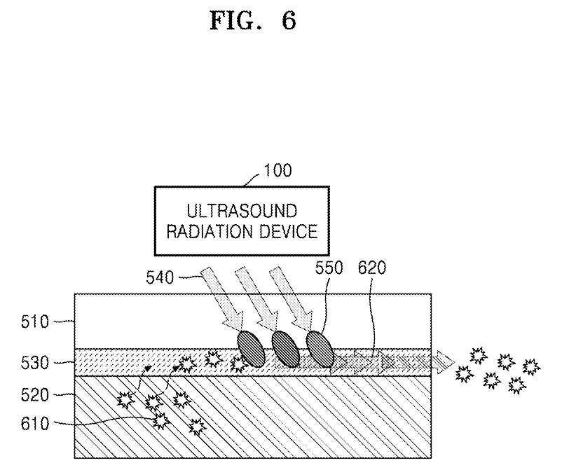

FIG. 6 is a diagram showing removal of waste materials by sweeping a focused ultrasound beam according to a method of radiating an ultrasound beam to accelerate lymphatic circulation, according to an embodiment.

The ultrasound radiation device 100 may radiate the focused ultrasound beam 540. The focused ultrasound beam 540 may be incident on the first medium 510. For example, the first medium 510 may be the skull. Part of the focused ultrasound beam incident on the first medium 510 may be refracted. The refracted ultrasound beam may be incident on the second medium 520. For example, the second medium 520 may be a substantial brain part including subarachnoid space. The rest of the ultrasound beam of the focused ultrasound beam 540 incident on the first medium 510, except the refracted ultrasound beam, may travel along the fluid 530. For example, the fluid 530 may be lymph flowing along the lymphatic vessels.

The focused ultrasound beam 540 incident on the first medium 510 may be refracted and split at the focus point 550. The focus point 550 may be located at the boundary between the first medium 510 and the second medium 520. The focus point 550 may be located at the fluid 530.

According to an embodiment a method of radiating an ultrasound beam to accelerate lymphatic circulation, waste materials 610 may be discharged by accelerating the movement of lymph by using transverse waves 620. The waste materials 610 may be generated in the second medium 520 and accumulated in the interior of the second medium 520. The waste materials 610 accumulated in the interior of the second medium 520 may be moved to the fluid 530. The waste materials 610 moved to the fluid 530 may be discharged to the outside by the movement of the fluid 530. When the movement of the lymph is accelerated by using the transverse waves 620, an amount of the waste materials 610 entering the inside of the lymphatic vessels may be increased. When the movement of the lymph is accelerated by using the transverse waves 620, the amount of the waste materials 610 discharged to the outside may be increased.

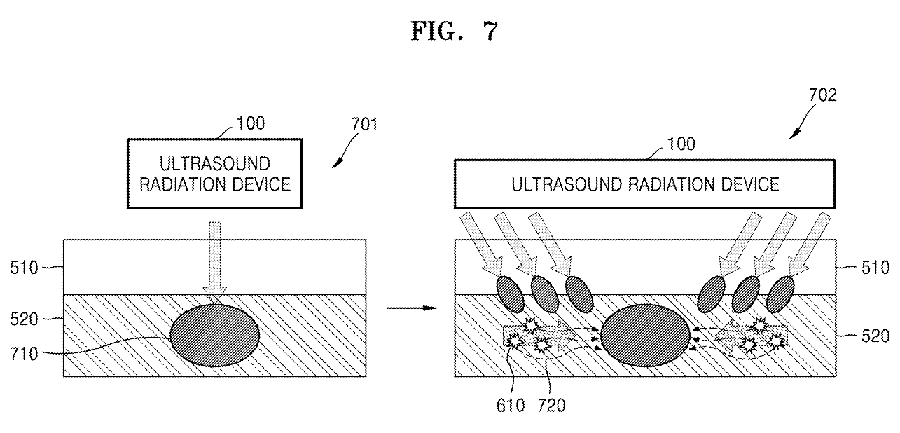

FIG. 7 is a diagram showing removal of waste materials by sweeping a focused ultrasound beam according to a method of radiating an ultrasound beam to accelerate lymphatic circulation, according to an embodiment.

The ultrasound radiation device 100 may radiate a focused ultrasound beam. The focused ultrasound beam may be incident on the first medium 510. For example, the first medium 510 may be the skull. The focused ultrasound beam incident on the first medium 510 may be incident on the second medium 520. For example, the second medium 520 may be a substantial brain part including subarachnoid space.

The focused ultrasound beam incident on the second medium 520 in a first situation 701 may be incident on a micro bubble 710. The micro bubble 710 may be a small space injected into the second medium 520. The micro bubble 710 may include gas. When ultrasound stimulation is applied to the micro bubble 710, the BBB may be opened.

The focused ultrasound beam incident on the second medium 520 in a second situation 702 may move the waste materials 610 in a direction indicated by arrows 720. The focused ultrasound beam incident on the second medium 520 may move the waste materials 610 in a direction indicated by arrows 720 in which the BBB is opened. The focused ultrasound beam incident on the second medium 520 may discharge the waste materials 610. The waste materials 610 moved to a portion where the BBB is opened may be discharged to the outside by blood vessels and the circulation of lymph.

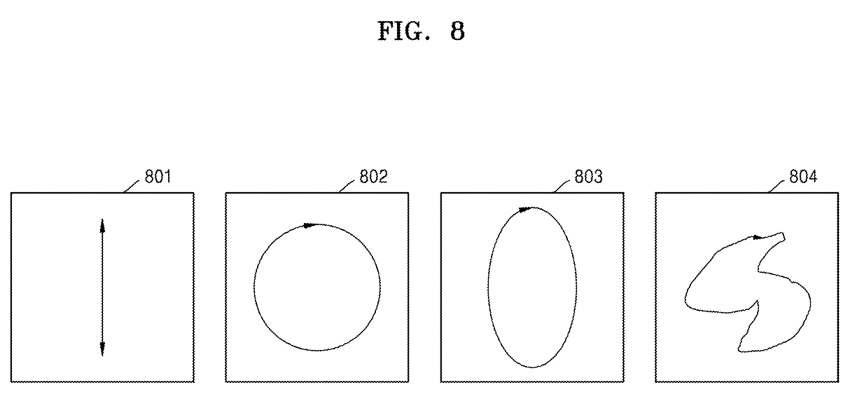

FIG. 8 is a diagram showing patterns formed when performing a sweeping operation of repeatedly scanning a focused ultrasound beam, according to an embodiment.

According to an embodiment, when a focused ultrasound beam is repeatedly scanned using the ultrasound radiation device 100, a sweeping operation may be performed while drawing a certain pattern. The certain pattern may include at least one of a linear reciprocal pattern 801, a circular pattern 802, an oval pattern 803, and a certain closed pattern 804.

When the certain pattern is the linear reciprocal pattern 801, the focused ultrasound beam may be scanned by repeatedly moving the ultrasound radiation device 100 while drawing a linear line with respect to a target point on the user's skull. When the certain pattern is the linear reciprocal pattern 801, an operation of imitating a sweeping motion using a broom may be performed. The sweeping motion using a broom may be an operation that applies force in one direction to sweep away dust, returns the broom to an original position of the broom, and then applies force in the one direction again to sweep away dust. Accordingly, when the certain pattern is the linear reciprocal pattern 801, the ultrasound radiation device 100 may scan the focused ultrasound beam only when moving in a specific direction. When the certain pattern is the linear reciprocal pattern 801, the ultrasound beam may not be scanned while the ultrasound radiation device 100 returns to the original position.

When the certain pattern is the circular pattern 802, the focused ultrasound beam may be scanned while drawing a circle in the clockwise direction or counterclockwise direction with respect to the target point on the user's skull.

When the certain pattern is the oval pattern 803, the focused ultrasound beam may be scanned while drawing an oval in the clockwise direction or counterclockwise direction with respect to the target point on the user's skull.

When the certain pattern is the closed pattern 804, the focused ultrasound beam may be scanned while drawing a polygon or closed curve with respect to the target point on the user's skull.

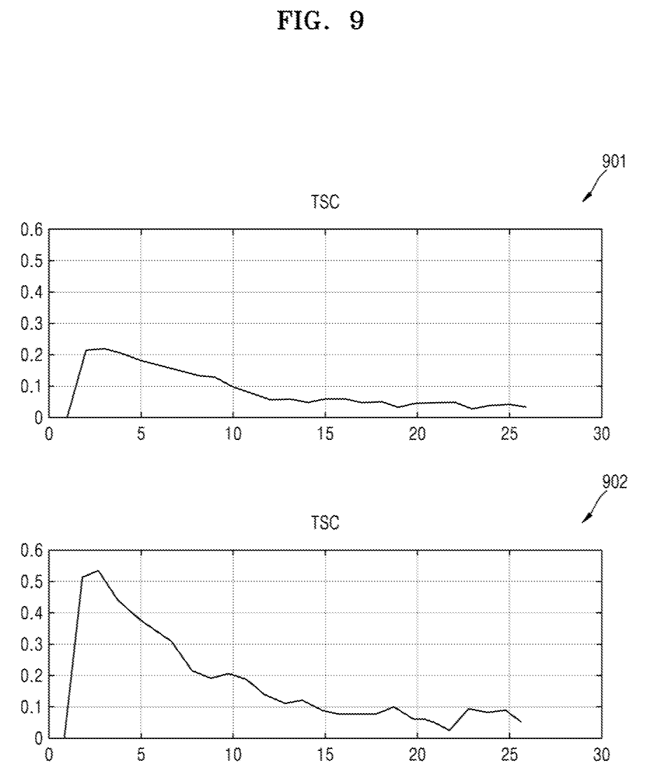

FIG. 9 includes graphs showing a comparison of circulation amount of lymph depending on the application of a method of radiating an ultrasound beam to accelerate lymphatic circulation, according to an embodiment.

In order to compare a first case 901 in which the method of radiating an ultrasound beam according to an embodiment is not applied with a second case 902 in which in which the method of radiating an ultrasound beam according to an embodiment is applied, contrast-enhanced image imaging may be performed using magnetic resonance imaging equipment. When data is obtained by performing contrast-enhanced image imaging on the first case 901 and the second case 902, data related to whether a CSF flow has increased may be obtained. For example, by performing sweeping of an ultrasound beam four times at one-hour intervals on rats injected with a gadolinium contrast agent into a cisterna magna region, known as an intermediate path of CSF circulation, data related to the increase in CSF flow may be obtained hourly for 24 hours.

When the method of radiating an ultrasound beam according to an embodiment is not applied as in the first case 901, it may be confirmed that a first contrast-enhanced signal is acquired in the CSF circulation path. When the method of radiating an ultrasound beam according to an embodiment is applied as in the second case 902, it may be confirmed that a second contrast-enhanced signal with a larger amplitude than the first contrast-enhanced signal is obtained by performing sweeping in the same CSF circulation path. Accordingly, when the method of radiating an ultrasound beam according to an embodiment is applied, it may be confirmed that an amount of CSF inflow increases and the discharge of waste materials is accelerated.

The disclosure aims to provide a technology that can accelerate lymphatic circulation by radiating an ultrasound beam and activate the lymphatic system to remove waste materials in tissues.

A method of radiating an ultrasound beam includes radiating a focused ultrasound beam into tissues of a human body while moving a position of the focused ultrasound beam, sweeping the focused ultrasound beam around an open area of a blood-brain barrier (BBB) inside the tissues of the human body, and activating circulation of lymph in a path through which waste materials in the tissues of the human body are discharged, by performing a sweeping operation of repeatedly scanning the focused ultrasound beam in a certain pattern.

In an embodiment, the focused ultrasound beam may be scanned in burst form.

In an embodiment, the focused ultrasound beam may be continuously radiated.

In an embodiment, an angle between the focused ultrasound beam and surface of the brain may be 25° to 55°.

In an embodiment, the focused ultrasound beam may generate transverse waves inside the brain.

In an embodiment, the waste materials may be discharged by accelerating movement of the lymph by using the transverse waves.

In an embodiment, the certain pattern may include at least one of a linear reciprocal pattern, a circular pattern, an oval pattern, and a certain closed pattern.

A method of radiating an ultrasound beam to accelerate lymphatic circulation, according to an embodiment may include radiating a focused ultrasound beam to a boundary portion between a skull and brain parenchyma; allowing the focused ultrasound beam to enter the boundary portion at a specific angle; radiating the focused ultrasound beam while moving a position of the focused ultrasound beam; and transmitting the focused ultrasound beam to an activation trigger area where a lymphatic system is activated, by performing a sweeping operation of repeatedly scanning the focused ultrasound beam in a certain pattern.

In an embodiment, the focused ultrasound beam may be scanned in burst form.

In an embodiment, the focused ultrasound beam may be continuously radiated.

In an embodiment, the specific angle may be 25° to 55°.

In an embodiment, the focused ultrasound beam may generate the transverse waves in the brain.

In an embodiment, the waste materials may be discharged by accelerating movement of the lymph by using the transverse waves.

In an embodiment, the certain pattern may include at least one of a linear reciprocal pattern, a circular pattern, an oval pattern, and a certain closed pattern.

According to the disclosure, the waste materials in tissues may be removed in a non-invasive manner by using the focused ultrasound beam.

The method according to an embodiment of the disclosure may be embodied as program instructions executable by various computer devices, and recorded on a computer-readable medium. The computer-readable medium may include a program command, a data file, a data structure, etc. solely or by combining the same. A program command recorded on the medium may be specially designed and configured for the disclosure or may be a usable one, such as computer software, which is well known to one of ordinary skill in the art to which the disclosure pertains to. Examples of the computer-readable recording medium include magnetic media such as hard disks, floppy disks, or magnetic tapes, optical media such as compact disc ROMs (CD-ROMs) or digital video discs (DVDs), magneto-optical media such as floptical disks, and hardware devices such as ROM, RAM, and flash memory, which are specially configured to store and execute program instructions. An example of a program command may include not only machine codes created by a compiler, but also high-level programming language executable by a computer using an interpreter.

An embodiment of the disclosure may be implemented in the form of a recording medium including computer executable instructions, such as a program module executed by a computer. A computer-readable storage medium may be a usable medium that is accessible by a computer and may include all of volatile and non-volatile media and separable and inseparable media. Furthermore, the computer-readable medium may include all of computer storage media and communication media. The computer storage media may include all of volatile and non-volatile media and separable and inseparable media, which are embodied by a certain method or technology for storing information such as computer-readable instructions, data structures, program modules, or other data. The communication media may typically include computer-readable instructions, data structures, program modules, or other data of a modulated data signal such as a carrier wave, or other transmission mechanism, and may also include information transmission media. Furthermore, some embodiments of the disclosure may also be implemented as a computer program or a computer program product that includes computer-executable instructions, such as a computer program that is executed by a computer.

Here, the machine-readable storage medium may be provided in the form of a non-transitory storage medium. Here, the term 'non-transitory storage medium' merely means that the storage medium does not refer to a transitory electrical signal (e.g., electromagnetic waves) but is tangible, and does not distinguish whether data is stored semi-permanently or temporarily on the storage medium. For example, the 'non-transitory storage medium' may include a buffer in which data is temporarily stored.

According to an embodiment of the disclosure, the methods according to various embodiments disclosed herein may be included in a computer program product and then provided. The computer program products may be traded as commodities between sellers and buyers. The computer program product may be distributed in the form of a machine-readable storage medium (e.g., a CD-ROM), or may be distributed online (e.g., downloaded or uploaded) through an application store or directly between two user devices (e.g., smartphones). In a case of online distribution, at least a portion of the computer program product (e.g., a downloadable app) may be temporarily stored in a machine-readable storage medium such as a manufacturer's server, an application store's server, or a memory of a relay server.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. A method of facilitating clearance of biological waste materials from a body of a subject, the method comprising:
radiating a focused ultrasound beam into tissues of the subject at a specific angle to a normal direction that is perpendicular to a surface of the tissues to convert longitudinal waves into transverse waves inside the tissues, wherein the transverse waves generate a directional acoustic radiation force;
moving the focused ultrasound beam along a predefined anatomical pathway adjacent to a lymphatic or perivascular structure; and
inducing anisotropic fluid movement to facilitate clearance of biological waste materials in the tissues.

2. The method of claim 1, further comprising performing a sweeping operation of repeatedly scanning the focused ultrasound beam in a certain pattern,
wherein the focused ultrasound beam is scanned in burst form.

3. The method of claim 1, wherein the focused ultrasound beam is continuously radiated.

4. The method of claim 1, wherein the specific angle is in a range of 25° to 55°.

5. (canceled)

6. (canceled)

7. The method of claim 1, further comprising performing a sweeping operation of repeatedly scanning the focused ultrasound beam in a certain pattern,
wherein the certain pattern comprises at least one of a linear reciprocal pattern, a circular pattern, an oval pattern, and a certain closed pattern.

8. A method of activating a lymphatic system in a brain, the method comprising:
radiating a focused ultrasound beam toward a boundary portion between a skull and brain parenchyma;
allowing the focused ultrasound beam to enter the boundary portion at a specific angle to a normal direction that is perpendicular to a surface of the boundary portion to convert longitudinal waves into transverse waves inside the boundary portion, wherein the transverse waves generate a directional acoustic radiation force;
moving the focused ultrasound beam along a predefined pathway corresponding to a lymphatic drainage route; and
activating a lymphatic system in the brain by inducing an anisotropic fluid movement along the lymphatic drainage route.

9. The method of claim 8, further comprising performing a sweeping operation of repeatedly scanning the focused ultrasound beam in a certain pattern,
wherein the focused ultrasound beam is scanned in burst form.

10. The method of claim 8, wherein the focused ultrasound beam is continuously radiated.

11. The method of claim 8, wherein the specific angle is in a range of 25° to 55°.

12. (canceled)

13. The method of claim 8, wherein waste materials are discharged by accelerating movement of lymph by using the transverse waves.

14. The method of claim 8 further comprising performing a sweeping operation of repeatedly scanning the focused ultrasound beam in a certain pattern,
wherein the certain pattern comprises at least one of a linear reciprocal pattern, a circular pattern, an oval pattern, and a certain closed pattern.

15. The method of claim 1, wherein the subject is a human or an animal.

16. The method of claim 1, wherein the body comprises a brain.

17. The method of claim 16, wherein the moving of the focused ultrasound beam includes sweeping the focused ultrasound beam around an open area of a blood brain barrier (BBB).

18. The method of claim 1, wherein the biological waste materials include at least one of amyloid-beta, tau protein, lactate, metabolic byproducts, inflammatory mediators, or interstitial waste substances.

19. The method of claim 1, wherein the body comprises at least one of an arm or a leg.
